(19) United States
(12) Patent Application Publication
(10) Pub. No.: US 2023/0024304 A1
(43) Pub. Date: Jan. 26, 2023

(54) A MULTICOMPONENT COMPOSITION COMPRISING A COLLAGEN, RESVERATROL AND ASTAXANTHIN AND THE USE THEREOF FOR THE TREATMENT OF TENDINOPATHIES

(71) Applicant: KOLINPHARMA S.P.A., Milano (MI) (IT)

(72) Inventors: Valeria CURTI, Milano (MI) (IT); Rita Paola PETRELLI, Milano (MI) (IT); Massimo MANTELLA, Milano (MI) (IT)

(21) Appl. No.: 17/785,004

(22) PCT Filed: Dec. 18, 2020
(86) PCT No.: PCT/IB2020/062191

§ 371 Date: Jun. 13, 2022

(30) Foreign Application Priority Data
Dec. 19, 2019 (IT) 102019000024907

(51) Int. Cl. A61K 31/05; A61K 31/07; A61K 31/375; A61K 36/704; A61K 9/14; A61K 9/10; A61P 19/02

(52) U.S. Cl. CPC A61K 31/05; A61K 31/07; A61K 31/375; A61K 36/704; A61K 9/14; A61K 9/10; A61P 19/02

(57) ABSTRACT

The present invention relates to compositions comprising collagen, resveratrol, astaxanthin, and vitamin C. Furthermore, the present invention relates to the use of said compositions for the treatment of tendinopathies and diseases affecting the joints or the musculoskeletal system.

The present invention relates to compositions comprising a mixture comprising or, alternatively, consisting of: (a) collagen; (b) at least one anti-inflammatory agent selected from (b-i) resveratrol and/or (b-ii) at least one curcuminoid; (c) at least one antioxidant selected from (c-i) astaxanthin and/or (c-ii) epigallocatechin gallate; and, optionally, said composition comprises (d) vitamin C and/or at least one acceptable pharmaceutical or food grade additive/or excipient. Furthermore, the present invention relates to the use of said compositions or mixtures for the treatment of tendinopathies and of diseases, symptoms or disorders related to said tendinopathies, such as disorders affecting the joints or the musculoskeletal system.

Tendon consists of a dense connective tissue that connects the skeletal muscle to bone and it transmits the strength produced by the movements. Tendons also have the function of stabilising joints and absorbing shocks, protecting muscles from damage. Tendons are rich in collagen fibres, present from about 65% to 80% with respect to the dry weight of the tendon. The most present collagen is of type I (95%), but collagen types III, V and X are also present in a smaller amount. Collagen has the function of providing the tendon with the strength to withstand high loads. Other tendon components, such as proteoglycans (e.g. decorin), glycoproteins and elastin, form the tendon matrix. These specific components confer resilience and biomechanical stability to the tendons.

Tendinopathy (or tendon disease) is a common disease of the musculoskeletal system, characterised by pain, swelling and limited function. Tendinopathies can be classified into:
enthesopathy, disease of the tendon insertion point;
peritendinitis, inflammation of the sheath surrounding the tendon;
tendinosis, tendon degeneration;
tendon rupture;
tenosynovitis, inflammation of the tendon bursa.

Several factors may cause tendon diseases and they can be divided into two main categories: extrinsic and intrinsic factors.

Among extrinsic factors, overload is considered one of the primary factors.

It is associated with sports activity and the damage related thereto.

Intrinsic factors include age, gender, anatomical abnormalities, systemic diseases (e.g. hyperlipidaemia) and genetic diseases.

20% of primary consultations with general practitioners are due to musculoskeletal problems. 30% of these relate to tendons and they are called tendinopathies.

Most injuries occur in areas near the joints, such as shoulders, elbows, knees and hips. There are tendons that suffer more from tendinopathies, such as: the supraspinatus tendon, the brachial biceps tendons, the forearm extensor and flexor tendons, the patellar ligament, the Achilles tendon and the posterior tibial tendons. Joint injuries accompanied by partial or total tendon tear occur up to 45% of all musculoskeletal injuries. Furthermore, tendon injuries occur with a frequency estimated between 30% and 50% of all injuries, and they may comprise accidental or sports trauma.

The role of inflammation in tendon degeneration and in the repair thereof has been subject of in-depth study and debate in recent years. Increasing evidence has attributed inflammation a fundamental role in the progression of the disease. It is assumed that chronic inflammation may guide degeneration prior to rupture and it may lead to fibrovascular cicatrisation during healing and thus, blocking of the inflammatory cascade is a primary objective for therapeutic intervention. The transcription factor NF-kB, known for its pro-inflammatory effect, was found to be increased in clinical samples of early-stage tendinopathies. Also IKK, a protein which if degraded (in case of inflammatory stimulus) allows NF-kB to enter the nucleus and, thus, to exert the pro-inflammatory power, has been found overexpressed in patients with rotator cuff problems. Furthermore, a study revealed an increase in NF-kB signal translation pathway in the subscapularis tendons of patients undergoing surgery for rotator cuff repair (Abraham et al., Science Translational Medicine, 2019). Studies of early-stage tendinopathies, including subscapularis, revealed an increase in the expression of NF-kB and proteins downstream transcribed by the latter and the activation of immune system cells. This inflammation persists in patients with chronic pain after subacromial decompressor treatment. Specifically, it has been shown that fibroblasts present in the microenvironment of the damaged tendon have a memory even 4 years after treatment, since they are more responsive to IL-1β in vitro stimulation and do not decrease the expression of IL-6 and IL-8 after cessation of the stimulus. This means that the pro-inflammatory stimulus persists and it is necessary to identify compositions capable of reducing or eliminating this degenerative process. Thus, the resolution of the inflammation by reducing or quenching the NF-kB transcription factor could be critical and fundamental for success in the outcome of tendon diseases. Attention is given to a protein upstream of NF-kB, i.e. IKK, which, if activated, leads to the ubiquitination of IKa and, thus, to the nuclear translocation of NF-kB. The inhibition of NF-kB nuclear translocation is therefore a positive event, given that it determines the negative modulation of the inflammation. As regards IL-1β, recent studies have shown that IL-1β causes a loss of tendon stem/progenitor cells in vitro phenotype and this is associated with decreased expression of tendon-related genes, such as scleraxis and tenomodulin. At the same time, the expression of collagen, biglycan and fibromodulin are sub-regulated. These data suggest that IL-1β inhibits the repair ability of tendons.

Besides propagating inflammation, inflammatory cascade also stimulates the production of other molecules, whose effects negatively impact the tendon disease, such as for example the synthesis of COX-2, metalloprotease 2 (MMP-2) and prostaglandin E2 (PGE2). All these molecules are known to be involved in tendon degeneration. Furthermore, inflammation leads to the production of substance P, a molecule involved in neurogenic inflammation. In tendons, small peripheral sensory neurons and the tenocytes can express the substance P. Painful stimuli and mechanical loads can activate the release of substance P.

Furthermore, the role of oxidative stress in tendon degeneration and in the repair thereof has been subject of in-depth study and debate in recent years. Tendon damage can lead to the formation of free radicals, such as hydrogen peroxide, and the release of mediators of inflammation, such as TNF-a, IL-1B and IL-6. Free radicals can activate MMPs and increase PGE2 expression, which degrade the tendon collagen matrix. This inflammatory process can activate the release of growth factors, for example TGF-B, which induces infiltration of tenocytes and fibroblasts, causing extensive reorganisation of collagen. The formation of type III collagen becomes greater with respect to the formation of type I collagen and this is due to the inhibitory effect of PGE2 on the synthesis of type I collagen, while the expression of type III collagen increases. Furthermore, oxidative stress can interfere with the healing process. The proliferation of type III collagen increases and the maturation of crosslinks between collagen fibres becomes smaller. The collagen components will tend to become fibrous, rigid, characterised by an uneven distribution of the load-bearing. These events cause a decrease in the tendon mechanical components by up to about 45%, making them vulnerable and prone to rapture.

To date, nonsteroidal anti-inflammatory drugs (NSAIDs) are commonly prescribed for the treatment of tendinopathies in order to reduce inflammation and, therefore, subsequent tissue damage. However, the use of NSAIDs is associated with numerous adverse events (side effects), including inhibition of proteoglycan synthesis and cell proliferation. Furthermore, the use of anti-inflammatory drugs alone does not allow to effectively treat the healing of a tendon affected by a disease, let alone the restoration of a damaged tendon to a healthy condition. In other words, the use of anti-inflammatory drugs cannot be considered as a targeted treatment for the preventive or curative treatment of tendinopathies.

Patent document WO 2014/184246 A1 discloses a dietary supplement comprising hydrolysed collagen, curcumin and green tea polyphenols comprising epigallocatechin gallate (EGCG) for use in the treatment of osteoarthritis and joint inflammation caused by osteoarthritis.

Thus, there is a high need to provide compositions or mixtures effective in the preventive and/or curative treatment of tendinopathies (or tendon diseases) and diseases, symptoms or disorders related to said tendinopathies (for example, diseases affecting the joints or the musculoskeletal system) that they are better than anti-inflammatory drugs and/or useful to reduce the amount of anti-inflammatory drugs used and, thus, the undesired effects of anti-inflammatory drugs.

Furthermore, there is a need to provide compositions or mixtures for an effective preventive and/or curative treatment of tendinopathies and diseases, symptoms or disorders related to said tendinopathies which are alternative or improved with respect to the products currently available in the prior art.

Furthermore, there is a need to provide compositions or mixtures for an effective preventive and/or curative treatment of tendinopathies and diseases, disorders or symptoms related to said tendinopathies, capable of allowing a rapid and almost total restoration of the functionality of the damaged tendons and, therefore, of the joints and/or portions of the musculoskeletal system involved in said tendon damage, that can be used by all classes of subjects (such as for example, sportsmen/sportswomen, the elderly and adults with normal healthy condition) and free of significant side effects.

Lastly, there is a need to provide compositions or mixtures for an effective preventive and/or curative treatment of tendinopathies and diseases, disorders or symptoms related to said tendinopathies which are easy to prepare and cost-effective.

Following extensive research and development activity, the Applicant addresses and solves the aforementioned and other needs resulting from the present description, by providing compositions (in short, compositions of the invention) comprising a mixture (in short, mixture of the invention) comprising or, alternatively, consisting of:
(a) collagen or collagen peptides, preferably hydrolysed collagen peptides, more preferably enzymatically hydrolysed collagen peptides;
(b) at least one anti-inflammatory agent selected from (b-i) resveratrol, preferably resveratrol from Polygonum cuspidatum or, alternatively, resveratrol from fermentation of Saccharomyces cerevisiae, and/or (b-ii) at least one curcuminoid, preferably extract of Curcuma longa comprising curcuminoids; (c) at least one antioxidant selected from (c-i) astaxanthin, preferably an extract of Haematococcus pluvialis comprising astaxanthin, and/or (c-ii) epigallocatechin gallate;

and, optionally, comprising (d) vitamin C and/or at least one acceptable pharmaceutical or food grade excipient-additive.

The compositions and mixtures subject of the present invention are highly effective in the curative and/or preventive treatment of tendinopathies (or tendon pathologies) and of diseases, symptoms or disorders related to said tendinopathies (for example, diseases, symptoms or disorders affecting the joints or the musculoskeletal system), and they can be an improvement with respect to treatments with anti-inflammatory agents or products of the prior art, and/or useful in decreasing the amount of anti-inflammatories used.

Said high efficacy of the compositions and mixtures of the present invention is due to the selected and specific association of the active components present in the compositions and mixtures and/or to their synergy, with particular reference to (a) collagen, (b) resveratrol and/or curcuminoid, (c) astaxanthin and/or epigallocatechin gallate and, optionally, (d) vitamin C, preferably with reference to the mixture comprising (a) collagen (b) resveratrol, (c) astaxanthin and (d) vitamin C.

As evidenced by clinical data, the anti-inflammatory and painkiller approach is not sufficient to act on the self-repair of the tissue given that tendons have a weak regenerative capacity. The addition of a structural component, such as collagen, and an antioxidant component to anti-inflammatory components, like in the compositions or mixtures subject of the present invention, effectively and rapidly supports the action of recovery of the tendon and/or joint. As a matter of fact, the primary objective of a product intended for tendinopathies should be the restoration of tendon functionality, and thus its main structural component, namely collagen.

Furthermore, said high efficacy of the compositions and mixtures subject of the present invention can result in a decrease in the effective dose to be administered to a subject in need, with respect to the compositions present in the prior art which show a lower effectiveness than those of the present invention. As a result, the compositions or mixtures of the present invention can therefore have an economic advantage and/or potentially a lower number of undesired effects, compared to said compositions present in the prior art.

In addition, said high efficacy of the compositions and mixtures subject of the present invention can result in the achievement of a greater effect considering the same amount or concentration of composition or mixture administered to a subject in need, as compared to the compositions present in the prior art.

In particular, the association and/or synergy of the active components present in the compositions and mixtures of the present invention (i.e. (a), (b), (c) and (d)) make said compositions and mixtures particularly effective in the prevention of tendinopathies, in the treatment of acute stages of tendinopathies and in the prevention of recurrence (or relapse) of said tendinopathies and, therefore, their becoming chronic.

Said high efficacy of the compositions and mixtures subject of the present invention can also result in a decrease in the onset of said compositions and mixtures after administration to a subject in need, with respect to the products of the prior art.

In the context of the present invention, the term “onset” is used to indicate the latency time between the administration of a composition/product and the pharmacological effect thereof. In other words, onset is the time that elapses from when the composition/product is administered up to the partial or total pharmacological effect.

Furthermore, the compositions and mixtures subject of the present invention are stable over time from the chemical/physical and organoleptic point of view.

Furthermore, the compositions and mixtures of the present invention do not have significant side effects, they are well tolerated and, therefore, can be administered to all subjects, in particular also to sportsmen/sportswomen and elderly subjects.

Lastly, the compositions and mixtures of the present invention are easy to prepare and cost-effective.

These and other objects which will be apparent from the detailed description that follows are achieved by the mixtures and by the compositions of the present invention thanks to the technical characteristics reported in the present description and claimed in the attached claims.

### DESCRIPTION OF THE FIGURES

FIG. 1 illustrates the comparison of the fluorescence between treatments in the presence and absence of hydrogen peroxide at concentration C4 (Table 5). Abbreviations: cel=control, Ast=astaxanthin, Col=collagen, Res=resveratrol and Vit=Vitamin C. Colours of the symbols: white: controls; grey: individual treatments; black: Mix. YES/NO in the titres indicates the possible treatment with hydrogen peroxide.

FIG. 2 describes the difference in the response of the individual treatments at concentration C4 with respect to the four treatments in mixture (MIX, C1-C4). Abbreviations: Ast=astaxanthin, Col=collagen, Res=resveratrol and Vit=Vitamin C. Colours of the symbols: orange: individual treatments; black: MIX.

FIG. 3 illustrates the comparison of the absorbance (expression index of IL-6) between controls in the presence and absence of IL-1β, and treatments with the individual active substances at concentration C4 (Table 5) and mixture thereof (1:8 MIX) in the presence of IL-1β. Abbreviations: Asx=astaxanthin, Coll=collagen, Res=resveratrol and Vit C=Vitamin C. Colours of the symbols: white: control; grey: individual treatments; black: MIX.

FIG. 4 illustrates the comparison of the absorbance (expression index of MMP-2) between controls in the presence and absence of IL-1β, and treatments with the individual active substances at concentration C4 (Table 5) and mixture thereof (1:8 MIX) in the presence of IL-1β. Abbreviations and Colours: as in FIG. 3.

### DETAILED DESCRIPTION OF THE INVENTION

Forming an object of the present invention is a composition (in short composition of the invention) comprising (I) and, optionally, (d) and/or (II), wherein (I) is a mixture M (in short mixture M of the invention) comprising or, alternatively, consisting of:
(a) collagen or collagen peptides;
(b) at least one anti-inflammatory agent selected from the group comprising or, alternatively, consisting of:
(b-i) resveratrol, (b-ii) at least one curcuminoid and mixtures thereof;
(c) at least one antioxidant selected from the group comprising or, alternatively, consisting of: (c-i) astaxanthin, (c-ii) epigallocatechin gallate and mixtures thereof; wherein (d) is L-ascorbic acid; and
wherein (II) is at least one acceptable pharmaceutical or food grade additive and/or excipient.

In a preferred embodiment of the composition subject of the present invention, said mixture M comprises or, alternatively, consists of: (a) collagen or collagen peptides; (b-i) resveratrol; and (c-i) astaxanthin; preferably said mixture M comprises or, alternatively, consists of: (a) collagen or collagen peptides; (b-i) resveratrol; (c-i) astaxanthin and (d) L-ascorbic acid.

In an embodiment of the present invention, the composition of the invention comprises said mixture M comprising or, alternatively, consisting of: (a) collagen or collagen peptides; (b-i) resveratrol; (c-i) astaxanthin; and, furthermore, said composition comprises (d) L-ascorbic acid and, optionally, (II).

In a preferred embodiment of the present invention, the composition of the invention comprises said mixture M comprising or, alternatively, consisting of:
(a) collagen or collagen peptides, preferably hydrolysed collagen peptides; more preferably enzymatically hydrolysed collagen peptides, even more preferably enzymatically hydrolysed collagen peptides having an average molecular weight comprised in the range from 0.3 kDa to 10 kDa (for example, 0.5 kDa, 1 kDa, 2 kDa, 4 kDa, 6 kDa, or 8 kDa, wherein kDa means kiloDalton), preferably from 1 kDa to 5 kDa, more preferably from 1.5 kDa to 3 kDa (for example about 2 kDa), and/or a protein content in a percentage comprised in the range from 70% to 99% (for example 75%, 80%, 85%, or 90%), preferably from 80% to 98%, more preferably about 91%-95% (% by weight with respect to the total weight of the collagen);
(b-i) resveratrol selected from (b-i.i) and (b-i.ii), wherein (b-i.i) is an extract of Polygonum cuspidatum titrated in resveratrol, preferably an extract of Polygonum cuspidatum titrated in resveratrol in a percentage comprised in a range from 80% to 99.5% (for example 85%, 90%, or 95%), preferably from 90% to 99.5%, more preferably to about 98% (% by weight with respect to the total weight of the extract), even more preferably a dry root extract of Polygonum cuspidatum; and wherein (b-i.ii) is resveratrol from fermentation of Saccharomyces cerevisiae, preferably titrated in resveratrol in a percentage comprised in a range from 70% to 99.5% (for example 75%, 80%, 85%, or 90%), preferably from 85% to 95%, more preferably to about 90% (% by weight with respect to the total weight of the fermentation product); and (c-i) astaxanthin, preferably astaxanthin in form of (c-i.i) extract of Haematococcus pluvialis, more preferably extract of Haematococcus pluvialis titrated in astaxanthin to a percentage comprised in a range from 0.1% to 10% (for example 0.5%, 1%, 1.5%, 2%, 2.5%, 3%, 3.5%, 4%, 4.5%, 5%, 6%, 7%, 8% or 9%), preferably from 0.5% to 7.5%, more preferably from 1% to 5% (for example about 2.5%) (% by weight with respect to the total weight of the extract); and, optionally, said composition further comprises (d) and/or (II).

In a more preferred embodiment of the present invention, the composition of the invention comprises said mixture M comprising or, alternatively, consisting of:
(a) enzymatically hydrolysed collagen peptides, even more preferably enzymatically hydrolysed collagen peptides having an average molecular weight comprised in the range from 1 kDa to 5 kDa, preferably from 1.5 kDa to 3 kDa (e.g. about 2 kDa), and/or a protein content in a percentage comprised in the range from 80% to 98%, more preferably about 91%-95% (% by weight with respect to the total weight of the collagen);
(b-i.i) extract of Polygonum cuspidatum titrated in resveratrol in a percentage comprised in a range from 80% to 99.5%, preferably from 90% to 99.5%, more preferably to about 98% (% by weight with respect to the total weight of the extract), even more preferably a dry root extract of Polygonum cuspidatum;
(c-i.i) extract of Haematococcus pluvialis titrated in astaxanthin in a percentage comprised in the range from 0.1% to 10%, preferably from 0.5% to 7.5%, more preferably from 1% to 5% (for example about 2.5%) (% by weight with respect to the total weight of the extract);
and, optionally, said composition further comprises (d) and/or (II).

In a more preferred alternative embodiment of the present invention, the composition of the invention comprises said mixture M comprising or, alternatively, consisting of:
(a) enzymatically hydrolysed collagen peptides, even more preferably enzymatically hydrolysed collagen peptides having an average molecular weight comprised in the range from 1 kDa to 5 kDa, preferably from 1.5 kDa to 3 kDa (e.g. about 2 kDa), and/or a by % protein content comprised in the range from 80% to 98%, more preferably about 90%-91% (% by weight with respect to the total weight of the collagen);
(b-i.ii) resveratrol from fermentation of Saccharomyces cerevisiae, preferably titrated in resveratrol in a % comprised in a range from 70% to 99.5%, preferably from 85% to 95%, more preferably to about 90% (% by weight with respect to the total weight of the resveratrol from fermentation);
(c-i.i) extract of Haematococcus pluvialis titrated in astaxanthin to a % comprised in the range from 0.1% to 10%, preferably from 0.5% to 7.5%, more preferably from 1% to 5% (for example about 2.5%) (% by weight with respect to the total weight of the extract).

Collagen is the main protein of the connective tissue in animals.

In an embodiment, said (a) collagen or collagen peptides is/are present in the composition or mixture of the invention (comprising (a), (b) such as (b-i) and/or (b-ii), (c) such as (c-i) and/or (c-ii) and, optionally, (d) and/or (e)) as hydrolysed collagen peptides; more preferably enzymatically hydrolysed collagen peptides, even more preferably enzymatically hydrolysed collagen peptides having an average molecular weight comprised in the range from 0.3 kDa to 10 kDa, preferably from 1 kDa to 5 kDa, more preferably from 1.5 kDa to 3 kDa (for example about 2 kDa), and a by % protein content comprised in the range from 70% to 99%, preferably from 80% to 98%, more preferably about 91%-95% (% by weight with respect to the total weight of the collagen). An example thereof is the commercial product TENDOFORTE® B manufactured by Gelita AG (Germany), having the characteristics reported in the experimental part.

Collagen or collagen peptides that can be used in the context of the present invention can be obtained according to standard procedures known to the man skilled in the art and/or reported in literature.

Resveratrol (3,5,4-trihydroxy-trans-stilbene; IUPAC name 5-[(E)-2-(4-hydroxyphenyl)ethenyl]benzene-1,3-diol, CAS 501-36-0) is a non-flavonoid phenol naturally produced by many plant species in defence of pathogenic agents such as bacteria or fungi.

Besides antioxidant and antithrombotic properties, resveratrol also has a high anti-inflammatory property. In particular, literature studies in human tenocytes have investigated the effect of resveratrol on IL-1β-mediated inflammatory signal and it was demonstrated that resveratrol is capable of suppressing IL-1β-induced activation of NF-kB and PI-3K in a dose and time-dependent manner.

In particular, treatment with resveratrol promotes the production of matrix components, such as type I collagen, type III collagen, tenomodulin and tenogenic transcription factor scleraxis, while inhibiting the synthesis of gene products involved with inflammation and apoptosis, thus exerting an anti-inflammatory effect.

In a preferred embodiment, said (b-i) resveratrol is present in the composition or mixture of the invention (comprising (a), (b) such as (b-i) and/or (b-ii), (c) such as (c-i) and/or (c-ii) and, optionally, (d) and/or (e)) as (b-i.i) extract of Polygonum cuspidatum (Siebold & Zucc.) titrated in resveratrol in a percentage comprised in a range from 80% to 99.5%, preferably from 90% to 99.5%, more preferably to about 98% (% by weight with respect to the total weight of the extract), even more preferably a dry root extract of Polygonum cuspidatum (herbaceous perennial plant, belonging to the genus Polygonum).

Said extract of Polygonum cuspidatum comprising resveratrol that can be used in the context of the present invention is advantageously an extract obtained and titrated according to standard procedures known to the man skilled in the art and/or reported in literature, for example, a dry hydroalcoholic with titration using the HPIC method. An example thereof is the commercial product Resveratrol 98% manufactured by Quimdis, having the characteristics reported in the experimental part.

In a preferred alternative embodiment, said (b-i) resveratrol is present in the composition or mixture of the invention (comprising (a), (b) such as (b-i) e/o (b-ii), (c) such as (c-i) and/or (c-ii) and, optionally, (d) and/or (e)) as (b-i.ii) resveratrol from fermentation of Saccharomyces cerevisiae, preferably titrated in resveratrol in a percentage comprised in a range from 70% to 99.5%, preferably from 85% to 95%, more preferably to about 90% (% by weight with respect to the total weight of the fermentation product).

Saccharomyces cerevisiae (Meyen ex E. C. Hansen, 1883) is an osmophilic unicellular organism belonging to the Kingdom of fungi and the family Saccharomycetaceae.

Resveratrol from fermentation of Saccharomyces cerevisiae that can be used in the context of the present invention is obtained according to standard procedures known to the man skilled in the art and/or reported in literature. An example thereof is the commercial product Resveratrol CWD90 at 90% manufactured by Evolva (Switzerland) having the characteristics reported in the experimental part.

Astaxanthin (c-i) is a carotenoid which has a high antioxidant property and anti-inflammatory property. The use of antioxidants, such as astaxanthin, during tendon injuries can help reduce oxidative stress due to the formation of ROS.

It is known that astaxanthin has a suppressive effect against expression of MMPs, PGE and TGF-B; said suppression can lead to the decrease in the fibrotic process.

Furthermore, the use of astaxanthin has been assumed to reduce the ratio of type III:type I collagen fibres and to increase the tensile strength of the tendon during the healing process.

The most important animal source of astaxanthin is krill (Euphausia superba) oil and the most important plant source of astaxanthin is a unicellular alga (microalga) of fresh water: Haematococcus pluvialis. Haematococcus pluvialis autonomously produces lipids containing natural astaxanthin when environmental conditions induce an oxidative stress. This occurs in the presence of saline water or high light radiation.

In a preferred embodiment, said (c-i) astaxanthin is present in the composition or mixture of the invention (comprising (a), (b) such as (b-i) and/or (b-ii), (c) such as (c-i) and/or (c-ii) and, optionally, (d) and/or (e)) as (c-i.i) extract of Haematococcus pluvialis, more preferably extract of Haematococcus pluvialis titrated in astaxanthin in a percentage comprised in the range from 0.1% to 10%, preferably from 0.5% to 7.5%, more preferably from 1% to 5% (for example about 2.5%) (% by weight with respect to the total weight of the extract).

Astaxanthin from Haematococcus pluvialis that can be used in the context of the present invention can be obtained according to standard procedures known to the man skilled in the art and/or reported in literature. An example thereof is the commercial product AstaREAL® EL25 manufactured by AstaReal, having the characteristics reported in the experimental part.

In a more preferred embodiment of the present invention, the composition comprises the mixture M comprising or, alternatively, consisting of:
(a) enzymatically hydrolysed collagen peptides, even more preferably enzymatically hydrolysed collagen peptides having an average molecular weight comprised in the range from 1 kDa to 5 kDa, preferably from 1.5 kDa to 3 kDa (e.g. about 2 kDa), and a by % protein content comprised in the range from 80% to 98%, more preferably about 91%-95% (% by weight with respect to the total weight of the collagen);
(b-i.i) extract of Polygonum cuspidatum titrated in resveratrol in a % comprised in a range from 80% to 99.5%, preferably from 90% to 99.5%, more preferably to about 98% (% by weight with respect to the total weight of the extract), even more preferably a dry root extract of Polygonum cuspidatum;
(c-i.i) extract of Haematococcus pluvialis titrated in astaxanthin to a % comprised in the range from 0.1% to 10%, preferably from 0.5% to 7.5%, more preferably from 1% to 5% (for example about 2.5%) (% by weight with respect to the total weight of the extract);

furthermore, said composition or mixture comprises (d) L-ascorbic acid and, optionally, said composition comprises (II).

L-ascorbic acid or ascorbic acid (also known as vitamin C, IUPAC name: (5R)-[(1S)-1,2-dihydroxyethyl]-3,4-dihydroxyfuran-2(5H)-one, example of CAS No. 50-81-7) is a naturally occurring organic compound with antioxidant properties.

Following damage to the musculoskeletal system, there is an intense production of ROS that negatively affects the microenvironment and the healing process, affecting cell viability and proliferation of collagen-producing cells. As a potent antioxidant, vitamin C is capable of neutralising ROS, relieving oxidative stress arising from inflammation.

In the context of the present invention, the terms “L-ascorbic acid”, “ascorbic acid” and “vitamin C” are synonyms and used interchangeably.

Advantageously, the compositions or mixtures subject of the present invention comprise, per daily intake or per single dosage unit, the active components at the following amounts:
(a) hydrolysed collagen peptides at an amount comprised in a range from 500 mg to 6,500 mg, preferably from 1,000 mg to 6,000 mg, more preferably from 2,000 mg to 5,500 mg (for example 2,500 mg or 5,000 mg);
(b-i) resveratrol at an amount comprised in a range from 10 mg to 600 mg, preferably from 30 mg to 400 mg, more preferably from 50 mg to 250 mg (for example, 100 mg or 200 mg);
(c-i) astaxanthin at an amount comprised in a range from 0.5 mg to 40 mg, preferably from 1 mg to 20 mg, more preferably from 2 mg to 10 mg (for example 3 or 4 mg or 6 or 8 mg);

and, if present, (d) L-ascorbic acid at an amount comprised in a range from 100 mg to 1,500 mg, preferably from 250 mg to 1.200 mg, more preferably from 400 mg to 1.100 mg (for example, 500 mg or 1,000 mg).

Said “daily intake” may be formulated into a single dosage unit (for example a capsule, a powder or granules in a sachet, a suspension or solution in a phial) or it can be formulated into two or three or more dosage units (for example, 2 capsules, 2 sachets, 2 phials).

Advantageously, the compositions or mixtures subject of the present invention comprise, per daily intake or per single dosage unit, the active components at the following percentages by weight with respect to the total weight of the compositions (i.e. mixture of the active components and additives/excipients):
(a) collagen or collagen peptides (preferably hydrolysed collagen peptides) in a percentage comprised in a range from 10% to 75%, preferably from 15% to 70%, more preferably from 20% to 60% (for example, 25% or 55%);
(b) resveratrol in a percentage comprised in a range from 0.1% to 6.5%, preferably from 0.25% to 5.5%, more preferably from 0.5% to 4% (for example, 0.75% or 1% or 1.65% or 2%);
(c) astaxanthin in a percentage comprised in a range from 0.01% to 0.5%, preferably from 0.02% to 0.25%, more preferably from 0.03% to 0.15% (for example, 0.040% or 0.090%);
and (d) L-ascorbic acid in a percentage comprised in a range from 2% to 18%, preferably from 3% to 16.5%, more preferably from 4% to 15% (for example, 5% or 13%).

Advantageously, the compositions or mixtures subject of the present invention comprise, per daily intake or per single dosage unit, active components at the following percentages by weight with respect to the total weight of the mixture of the active components (i.e. (a), (b), (c) and (d)):
(a) collagen or collagen peptides (preferably hydrolysed) in a percentage comprised from 50% to 90% (for example about 60%, 70%, 80%, 90% or 95%), preferably from 75% to 85% (for example about 80%);
(b) resveratrol in a percentage comprised from 0.5% to 8% (for example about 1%, 1.5%, 2%, 2.5%, 3%, 3.5%, 4%, 5%, 6% or 7%), preferably from 2% to 4% (for example about 3.1%-3.3% or 2.3%-2.4%);
(c) astaxanthin in a percentage comprised from 0.05% to 2% (for example about 0.1%, 0.15%, 0.20%, 0.30%, 0.50%, 0.80%, 1%, 1.5%), preferably from 0.05% to 0.5% (for example about 0.09%-0.1% or 0.12%-0.14%);
and (d) L-ascorbic acid in a percentage comprised from 5% to 30% (for example about 8%, 10%, 12%, 14%, 16%, 18%, 20% or 25%), preferably from 12% to 18% (for example about 15.5%-16.5%).

Advantageously, the mixtures or compositions subject of the present invention comprise, per daily intake or per single dosage unit, the active components in a ratio by weight (or parts by weight) as defined below: [(a) collagen or collagen peptides from 500 to 50]: [(b) resveratrol from 5 to 0.5]: [(c) astaxanthin from 0.25 to 0.025]: [(d) L-ascorbic acid from 50 to 5]; preferably [(a) collagen or collagen peptides from 200 to 50]: [(b) resveratrol from 2 to 0.5]: [(c) astaxanthin from 0.1 to 0.025]: [(d) L-ascorbic acid from 20 to 4]; for example about: [(a) collagen or collagen peptides 100]: [(b) resveratrol 1]: [(c) astaxanthin from 0.05]: [(d) L-ascorbic acid 8.5-9].

Curcuminoids or curcumins are defined as the chemical compounds that form the pigment extracted from Curcuma longa, the analogues thereof or derivatives of natural and synthetic origin. Curcuminoids have anti-inflammatory and antioxidant properties.

In an embodiment of the composition or mixture of the invention (comprising (a), (b) such as (b-i) and/or (b-ii), (c) such as (c-i) and/or (c-ii) and, optionally, (d) and/or (e)) said (b) comprises or, alternatively, consists of said (b-ii) at least one curcuminoid (E100 according to the European directives), in addition to (b-i) or in substitution to (b-i).

Preferably, said (b-ii) at least one curcuminoid is an extract of Curcuma longa (L., 1753) (alternatively referred to as curcuma or saffron of the indies or turmeric) titrated in curcuminoids, such as for example curcumin, desmethoxycurcumin (DMC) and bis-desmethoxycurcumin (BDMC), in a % by weight comprised in the range from 15% to 95%, preferably from 20% to 70%, more preferably from 25% to 45% (for example about 35%), with respect to the total weight of extract.

An example thereof is the commercial product CurQfen NF manufactured by Spiceuticals, such as extract of Curcuma longa and de-bacterialised fenugreek powder rich in soluble dietary fibre, having the following technical characteristics: form: powder, particle size: >90% passes through 60 mesh sieve; solubility: 0.03 g/L in H₂O; density 0.30-0.70 g/ml (bulk), 0.40-0.80 g/ml (tapped); content: total curcuminoids (curcumin, DMC, BDMC) NLT 35% (HPLC), fenugreek fibres NLT 30% (internal method based on AOAC method); moisture content NMT 5% (USP 29<921> KF).

Advantageously, when the composition or mixture of the invention (comprising (a), (b) such as (b-i) and/or (b-ii), (c) such as (c-i) and/or (c-ii) and, optionally, (d) and/or (e)) comprises said (b-ii) curcuminoids (for example, a compound comprising curcuminoids in a percentage by weight comprised in the range from 25% to 45%, preferably at 35%), (b-ii) is comprised in a daily intake or in a dosage unit in an amount comprised in the range from 30 mg to 300 mg, preferably from 40 mg to 250 mg, more preferably from 50 mg to 200 mg.

Epigallocatechin gallate (in short EGCG) is a type of catechin (example of CAS No. 989-51-5) abundant in tea, particularly green tea, with high antioxidant properties.

In an embodiment of the composition or mixture of the invention (comprising (a), (b) such as (b-i) and/or (b-ii), (c) such as (c-i) and/or (c-ii) and, optionally, (d) and/or (e)) said (c) comprises or, alternatively, consists of said (c-ii) epigallocatechin gallate, in addition to (c-i) or in substitution to (c-i). Said (c-ii) used in the present invention may be an extract of a tea plant containing epigallocatechin gallate, preferably an extract of Camelia sinensis (L.) Kuntze comprising EGCG in a percentage by weight comprised in the range from 10% to 95%, preferably from 20% to 80%, more preferably from 25% to 50% (for example about 30%), with respect to the total weight of extract.

Said extract of Camelia sinensis comprising EGCG that can be used in the context of the present invention is, advantageously, an extract obtained and titrated according to standard procedures known to the man skilled in the art and/or reported in literature, for example, a dry leaf extract of Camelia sinensis with hydroalcoholic extraction solvent (e.g. ethanol/water) with titration in EGCG by means of spectroscopic method, for example HPLC.

An example thereof is the commercial product “Green tea 95% polyphenols/65% catechin” manufactured by EPO Istituto Farmochimico Fitoterapico S.r.l. Italia (CAS No.: 84650-60-2) by extracting leaves of Camelia sinensis with extraction solvent: ethanol 70/water 30 and having the following characteristics: excipients: maltodextrin, particle size not less than 90% passes through 300 microns; Titre: catechins (HPLC meth.)>=65.0% w/w, epigallocatechin gallate (EGCG): >=30.0% w/w, caffeine: <=5.0% w/w, epicatechin gallate (ECG): >=8.0% w/w; Titre: Total polyphenols (Folin—ciocalteau spectrophotometric meth.)>=95.0% w/w; Relative density: 350-650 g/I; Loss on drying: <=5.0% w/w; pH: 3.0-6.0.

Advantageously, when the composition or mixture of the invention (comprising (a), (b) such as (b-i) and/or (b-ii), (c) such as (c-i) and/or (c-ii) and, optionally, (d) and/or (e)) comprises said (c-ii) epigallocatechin gallate, (c-ii) is comprised in a daily intake or in a dosage unit in an amount comprised in the range from 50 mg to 300 mg, preferably from 100 mg to 250 mg, more preferably from 150 mg to 200 mg.

Lastly, in an embodiment of the composition or mixture of the invention, comprising (a), (b) such as (b-i) and/or (b-ii), (c) such as (c-i) and/or (c-ii), and, optionally, (d), said composition or mixture further comprises (e) myrrh.

Myrrh is an aromatic gum resin, extracted from a tree or shrub of the genus Commiphora, of the family Burseraceae. Literature studies have shown that myrrh is a potent analgesic part of the family of sesquiterpenes, molecules that have the same mechanism of action as cannabinoids.

The compositions of the present invention (comprising said mixture M (i.e. (a)+(b)+(c)) and, optionally, (d) and/or (e)) may comprise (II) at least one acceptable pharmaceutical or food grade additive and/or excipient, i.e. a substance devoid of therapeutic activity suitable for pharmaceutical or food use. In the context of the present invention, the acceptable additives and/or excipients for pharmaceutical or food use comprise all the ancillary substances known to the man skilled in the art for the preparation of compositions in solid, semi-solid or liquid form, such as, for example, diluents, solvents (including water), solubilisers, acidifiers, thickeners, sweeteners, flavour enhancers, colorants, sweeteners, lubricants, surfactants, preservatives, stabilisers, pH stabilising buffers and mixtures thereof. Preferably, said at least one acceptable pharmaceutical or food grade additive and/or excipient are selected from: magnesium stearate, silicon dioxide, dicalcium phosphate, microcrystalline cellulose and/or film-forming agents.

Embodiments (in short FR) of the composition of the present invention are reported below, comprising the active components (a), (b-i) such as (b-i.i) or (b-i.ii), (b-ii), (c-i) such as (c-i.i), (c-ii), (d) and (e) as reported in the present description:
FRa: (a), (b-i), (c-i) and, optionally, (d) and/or (e);
FRb: (a), (b-ii), (c-i) and, optionally, (d) and/or (e);
FRc: (a), (b-i), (b-ii), (c-i) and, optionally, (d) and/or (e);
FRd: (a), (b-ii), (c-i) and, optionally, (d) and/or (e);
FRe: (a), (b-ii), (c-ii) and, optionally, (d) and/or (e);
FRf: (a), (b-i), (b-ii), (c-ii) and, optionally, (d) and/or (e);
FRg: (a), (b-i), (b-ii), (c-i), (c-ii) and, optionally, (d) and/or (e);
FRh: (a), (b-i), (c-i), (c-ii) and, optionally, (d) and/or (e);
FRi: (a), (b-ii), (c-i), (c-ii) and, optionally, (d) and/or (e).

According to an embodiment, the compositions or mixtures M subject of the present invention do not comprise an active component of the non-vitamin type with antioxidant and/or anti-inflammatory properties, characterised in that said component contains at least one phenylethanoid, wherein said phenylethanoid contains at least one acteoside and/or isoacteoside.

According to an embodiment, the compositions or mixtures M subject of the present invention do not comprise bromelain and/or an extract of Boswellia (Roxb. ex Colebr.), plant belonging to the family of Burseraceae.

The compositions or mixtures M subject of the present invention can be in a liquid form, such as solution, biphasic liquid system, dispersions or suspension of a solid in a liquid (for example to be formed at the time of use), in a semisolid form, such as gel, cream or foam, or in a solid form, such as powder, granules, microgranules, flakes, tablets or capsules.

Preferably, the compositions or mixtures of the invention are formulated in a form suitable for oral (or gastroenteric), sublingual, buccal administration, for example in liquid form, in solid form as such or in mouth-soluble (or mouth-dispersible, which dissolve in the oral cavity) solid form.

In a preferred embodiment of the present invention, the compositions or mixtures of the invention are in solid form, preferably in the form of granules or powder for oral suspension (for example to be formed at the time of use) or mouth-soluble (for example, packaged as sachets containing the powder or granules).

In a preferred alternative embodiment of the present invention, the compositions or mixtures of the invention are in liquid form for oral use (for example, packaged as single-dose drinkable phials).

The composition of the invention, comprising said mixture subject of the present invention according to any of the described embodiments, may be a pharmaceutical composition, a medical device composition or a dietary supplement.

In the context of the present invention, the expression “medical device” is used in the meaning according to the Italian Legislative Decree n° 46 dated 24 Feb. 1997 or according to the new Medical Device Regulation (EU) 2017/745 (MDR).

Forming an object of the present invention are the compositions of the present invention and the mixtures M of the present invention (comprising (a), (b) such as (b-i) and/or (b-ii), and (c) such as (c-i) and/or (c-ii), and, optionally, (d) and/or (e) and/or additives/excipients) according to any one of the embodiments of the present invention, for use as medicament.

Forming an object of the present invention are the compositions of the present invention and the mixtures M of the present invention (comprising (a), (b) such as (b-i) and/or (b-ii), and (c) such as (c-i) and/or (c-ii), and, optionally, (d) and/or (e) and/or additives/excipients, according to any one of the embodiments of the present invention) for use in a method for the preventive and/or curative treatment of a tendinopathy and/or a disease, disorder or symptom related to said tendinopathy, in a subject in need, wherein said treatment method provides for the administration of an effective dose of a composition or mixture M subject of the present invention to said subject.

Advantageously, said tendinopathy is selected from: enthesopathy, peritendinitis, tendinosis, tendon rupture, tenosynovitis, and a disease, disorder or symptom related thereto.

Furthermore, disease, disorder or symptom related to said tendinopathy may be selected from:
disease, disorder or symptom affecting the joints, preferably inflammation of the joints and/or pain of the joints;
disease, disorder or symptom affecting the musculoskeletal system.

Forming an object of the present invention are a method for the preventive and/or curative treatment of tendinopathy and/or a disease or symptom related to said tendinopathy, in a subject in need, wherein said treatment method provides for the administration of a therapeutically effective amount of a composition or mixture M subject of the present invention to said subject.

Forming an object of the present invention is the non-therapeutic use of the compositions and mixtures M of the present invention in healthy subjects.

For the sake of clarity, in order to achieve the subject of the present invention, the components (or active components) (a), (b), (c), (d) and (e) of the composition of the invention can be administered simultaneously or separately, (preferably in a time interval from 5 minutes to 60 minutes) and in any order; preferably (a), (b), (c), (d) and (e) are administered to a subject simultaneously, even more preferably in a single composition to obtain a more rapid effect and for ease of administration.

When the active components of the invention are administered in a single composition, said single composition corresponds to the composition of the present invention.

In the context of the present invention, the term “subject/s” is used to indicate human or animal subjects, preferably mammals (e.g. pets such as dogs, cats, horses, sheep or cattle). Preferably, the compositions of the invention are for use in treatment methods for human subjects.

Unless specified otherwise, the expression composition or mixture or other comprising a component at an amount “comprised in a range from x to y” is used to indicate that said component can be present in the composition or mixture or other at all the amounts present in said range, even though not specified, extremes of the range comprised.

Unless specified otherwise, the content of a component in a composition or mixture refers to the percentage by weight of that component with respect to the total weight of the composition or mixture.

Unless specified otherwise, the indication that a composition or mixture or other “comprises” one or more components means that other components can be present besides that or those specifically indicated and the indication that a composition or mixture or other “consists” of determined components means that the presence of other components is excluded.

Aspects of the present invention (embodiments FRa) are reported below:
FRa1. A composition comprising
(I) a mixture M comprising, or alternatively, consisting of:
   (a) collagen or collagen peptides;
   (b) at least one anti-inflammatory agent selected from the group comprising or, alternatively, consisting of: (b-i) resveratrol, (b-ii) at least one curcuminoid and mixtures thereof;
   (c) at least one antioxidant selected from the group comprising or, alternatively, consisting of: (c-i) astaxanthin, (c-ii) epigallocatechin gallate and mixtures thereof;
and, optionally, said composition comprises (II) at least one acceptable pharmaceutical or food grade additive and/or excipient.
FRa2. The composition according to FRa1, wherein said mixture M comprises or, alternatively, consists of:
(a) collagen peptides, preferably hydrolysed collagen peptides; (b-i) resveratrol; (c-i) astaxanthin.
FRa3. The composition according to FRa1 or FRa2, wherein said (a) collagen peptides are hydrolysed collagen peptides; preferably (a) enzymatically hydrolysed collagen peptides having an average molecular weight comprised in the range from 0.3 kDa to 10 kDa, preferably from 1 kDa to 5 kDa, more preferably from 1.5 kDa to 3 kDa.
FRa4. The composition according to any one of FRa1-FRa3, wherein said composition further comprises (d) ascorbic acid; preferably wherein said composition comprises (d) ascorbic acid and said mixture M comprising or, alternatively, consisting of: (a) hydrolysed collagen peptides, (b-i) resveratrol, (c-i) astaxanthin.
FRa5. The composition according to any one of FRa1-FRa4, wherein said (b-i) resveratrol is selected from:
   (b-i.i) extract of Polygonum cuspidatum titrated in resveratrol in a % by weight comprised in a range from 80% to 99.5% with respect to the total weight of the extract, preferably from 90% to 99.5%, more preferably about 98%; preferably wherein said (b-i.i) is a dry root extract of Polygonum cuspidatum; and
   (b-i.ii) resveratrol from fermentation of Saccharomyces cerevisiae comprising resveratrol in a % comprised in a range from 70% to 99.5% with respect to the total weight, preferably from 85% to 95%, more preferably at about 90%.
FRa6. The composition according to any one of FRa1-FRa5, wherein said (c-i) astaxanthin consists of: (c-i.i) extract of Haematococcus pluvialis extract titrated in astaxanthin in a by weight % comprised in the range from 0.1% to 10% with respect to the total weight of extract, preferably from 0.5% to 7.5%, more preferably from 1% from 5%.
FRa7. The composition according to any one of FRa1-FRa6, wherein said composition is formulated for oral use; preferably in powdered or granular solid form or, alternatively, in liquid form of suspensions or solutions.
FRa8. The composition according to any one of FRa1-FRa7, wherein said composition is for use as medicament.
FRa9. The composition for use according to FRa8, wherein said composition is for use in a method for the preventive and/or curative treatment of a tendinopathy and/or a disease, disorder or symptom related to said tendinopathy, in a subject in need;
preferably wherein said tendinopathy is selected from: enthesopathy, peritendinitis, tendinosis, tendon rupture and/or tenosynovitis.
FRa10. The composition for use according to FRa9, wherein said disease, disorder or symptom related to said tendinopathy is selected from:
   disease, disorder or symptom affecting the joints, preferably inflammation of the joints and/or pain of the joints;
   disease, disorder or symptom affecting the musculoskeletal system.

Further embodiments of the present invention (FRb) are reported below:
FRb1. A composition comprising
(I) a mixture M comprising, or alternatively, consisting of:
   (a) collagen or collagen peptides;
   (b-i) resveratrol;
   (c-i) astaxanthin;
   (d) ascorbic acid;
and, optionally, said composition comprises
(II) at least one pharmaceutical or food grade additive and/or excipient.
FRb2. The composition according to FRb1, wherein said (a) collagen peptides are hydrolysed collagen peptides; preferably (a) enzymatically hydrolysed collagen peptides.
FRb3. The composition according to FRb1 or FRb2, wherein said (a) collagen or collagen peptides have an average molecular weight comprised in the range from 0.3 kDa to 10 kDa, preferably from 1 kDa to 5 kDa, more preferably from 1.5 kDa to 3 kDa.
FRb4. The composition according to any one of FRb1-FRb3, wherein said (b-i) resveratrol consists of (b-i.i) an extract of Polygonum cuspidatum titrated in resveratrol in a percentage by weight comprised from 80% to 99.5% with respect to the total weight of the extract, preferably from 90% to 99.5%, more preferably about 98%; preferably wherein said (b-i.i) is a dry root extract of Polygonum cuspidatum.
FRb5. The composition according to any one of FRb1-FRb3, wherein said (b-i) resveratrol consists of (b-i.ii) a resveratrol from fermentation of Saccharomyces cerevisiae comprising resveratrol in a percentage comprised from 70% to 99.5% with respect to the total weight of resveratrol from fermentation, preferably from 85% to 95%, more preferably to about 90%.
FRb6. The composition according to any one of FRb1-FRb5, wherein said (c-i) astaxanthin consists of: (c-i.i) extract of Haematococcus pluvialis titrated in astaxanthin in a percentage by weight comprised from 0.1% to 10% with respect to the total weight of extract, preferably from 0.5% to 7.5%, more preferably from 1% to 5%.
FRb7. The composition according to any one of FRb1-FRb6, wherein said mixture M comprises in percentages by weight with respect to the total weight of the mixture M consisting of (a), (b), (c) and (d):
(a) collagen or collagen peptides in a percentage comprised from 50% to 90%, preferably from 75% to 85%;
(b) resveratrol in a percentage comprised from 0.5% to 8%, preferably from 2% to 4%;
(c) astaxanthin in a percentage comprised from 0.05% to 2%, preferably from 0.05% to 0.5%;
and (d) L-ascorbic acid in a percentage comprised from 5% to 30%, preferably from 12% to 18%.
FRb8. The composition according to any one of FRb1-FRb7, wherein a dosage unit of said composition comprises in a ratio by weight [(a) collagen peptides 500 to 50]: [(b) resveratrol from 5 to 0.5]: [(c) astaxanthin from 0.25 to 0.025]: [(d) L-ascorbic acid from 50 to 5];
preferably [(a) collagen or collagen peptides from 200 to 50]: [(b) resveratrol from 2 to 0.5]: [(c) astaxanthin from 0.1 to 0.025]: [(d) L-ascorbic acid from 20 to 4];
more preferably [(a) collagen or collagen peptides 100]: [(b) resveratrol 1]: [(c) astaxanthin from 0.05]: [(d) L-ascorbic acid 8.5-9].
FRb9. The composition according to any one of FRb1-FRb8, wherein said composition is formulated for oral use.
FRb10. The composition according to FRb9, wherein said composition is in powdered or granular solid form or, alternatively, in liquid form of suspensions or solutions.
FRb11. The composition according to any one of FRb1-FRb10 for use as medicament.
FRb12. The composition according to any one of FRb1-FRb10 for use in a method for the preventive and/or curative treatment of a tendinopathy, enthesopathy, peritendinitis, tendinosis, tendon rupture, tenosynovitis and/or symptoms related thereto, in a subject in need.
FRb13. The composition for use according to FRb12, wherein said symptoms related to said tendinopathy are selected from joint inflammation and/or joint pain.

### EXPERIMENTAL PART (I)

Material

(I) Compositions in Powdered or Granular Solid Form for Oral Suspension (Sachets)

Compositions 1 and 2 are compositions according to the invention for oral use formulated in powdered or granular solid form for oral suspension (packaged in sachets), as reported in Table 1 and Table 2.

TABLE 1

| Components | mg/dose (1-2 sachets) |
|---|---|
| hydrolysed collagen peptides | 2500 mg-5000 mg |
| resveratrol (98%) | 100 mg-200 mg |
| astaxanthin | 3 or 4 mg 6 or 8 mg |
| vitamin C (ascorbic acid)* | 500 mg-1000 mg |
| Additives and/or excipients | q.s. |
| Total Weight | 45000 mg-9000 mg |

*VNR %/1 sachet = 1250

TABLE 2

| Components | mg/dose (1-2 sachets) |
|---|---|
| hydrolysed collagen peptides | 2500 mg-5000 mg |
| resveratrol (90%) | 75 mg-150 mg |
| astaxanthin | 3 or 4 mg 6 or 8 mg |
| vitamin C (ascorbic acid)* | 500 mg-1000 mg |
| Additives and/or excipients | q.s. |
| Total Weight | 45000 mg-9000 mg |

*VNR %/1 sachet = 1250

Additives and/or excipients of Table 1, 2, 3 and 4 are, for example, at least one acidifier, anti-caking agent, sweetener, flavour enhancement agent, colorant and sweetening agent.

Specific examples of the Compounds 1 and 2 are represented by the Compounds 1a (Table 1a) and 2a (Table 2a), respectively, wherein:
astaxanthin is present in form of Haematococcus pluvialis titrated to about 2.5% in astaxanthin (for example, ASTAREAL® EL25 of AstaReal).
resveratrol is present in the form of:
i) resveratrol extracted from Polygonum cuspidatum (Siebold & Zucc.) (for example, Resveratrol 98% manufactured by Quimdis s.a.s. France), or, ii) resveratrol (or trans-resveratrol) from fermentation of Saccharomyces cerevisiae (for example, RESVERATOLO CWD90 manufactured by Evolva);
   hydrolysed collagen peptide, having an average molecular weight of about 2 kDa, obtained by enzymatic hydrolysis of collagen (for example, TENDOFORTE® B manufactured by Gelita AG).

The technical characteristics of the aforementioned commercial products are illustrated below.

AstaREAL® EL25 (manufactured by AstaReal) is a free flow red powder containing 2.5% natural astaxanthin. The powder is produced by encapsulating AstaREAL® L10 in modified starch. AstaREAL® L10 is an oil rich in astaxanthin produced by the extraction with supercritical carbon dioxide in the absence of solvent of crushed and spray-dried aplanospores of the green microalgae Haematococcus pluvialis.

Composition: modified starch: 73.5%; AstaREAL® L10: 25%; astaxanthin: 2.5%-2.7% (on total carotenoids 2.6%-2.9%) including as monoesters 1.9%, as diesters 0.5% and as free form 0.1%.

Resveratrol 98% manufactured by Quimdis s.a.s. France, such as resveratrol extracted from Polygonum cuspidatum, has the following technical characteristics: CAS N° 501-36-0; Particle size: >95% passes through 80 mesh sieve; Solubility: 0.03 g/L in H₂O; Density>0.40 g/ml (bulk), >0.050 g/ml (tapped); Content≥98% (HPLC); Loss on drying ≤5% (CP); Hydroalcoholic extraction (e.g. EtOH: H₂O).

RESVERATROLO CWD 90 manufactured by Evolva, such as resveratrol from fermentation of Saccharomyces cerevisiae, has the following technical characteristics: Composition: 90% from trans-resveratrol produced by fermentation and 10% of excipients; Form: powder dispersible in cold water; Dispersion: dispersion of 560 mg in 100 ml of water passes through a 720 micron screen; Titre (on the anhydrous)>90.0%; IR Identification: >90% corresponds to the internal standard; Grain size: particles <10 microns >=10.00%; Average particle size <50.00 μm; Median grain size <45 μm.

TENDOFORTE® B (specific Bioactive Collagen Peptides® manufactured by Gelita AG, Germany) has the following technical characteristics: highly bioactive collagen peptides is about 2000 g/mol (about 2 kDa); collagen hydrolysed through enzymatic hydrolysis; protein content 91%-95% (technique: Kjeldahl (N×5.55)); Dry substance (105° C., 16 hours) 90%-96% (technique: GME); ashes (550° C.) 1.5% (technique: USP/GME); pH (10%, room temperature) 5.5-6.5 (technique: potentiometer); Viscosity (10%, 25° C.) 1.10-2.00 mPas (technique: method GELITA).

TENDOFORTE® B is pure collagen protein with an extremely low mineral content and it is devoid of fats, cholesterol, carbohydrates and dietary fibres.

TABLE 1a

| Components | mg/dose (1-2 sachets) | % |
|---|---|---|
| hydrolysed collagen peptides | 2500 mg-5000 mg | 55.55 |
| resveratrol from Polygunum cuspidatum | 100 mg-200 mg | 2.27 |
| (titrated to 98% in resveratrol) | | |
| Haematococcus pluvialis titrated to 2.5% in | 120 or 160 mg-240 or 320 mg | 3.55 |
| astaxanthin | (astaxanthin 3 or 4 mg 6 or 8 mg) | |
| vitamin C: ascorbic acid | 500 mg-1000 mg | 13.33 |
| acidifier: citric acid | 100 mg-200 mg | 2.22 |
| anti-caking agent: silicon dioxide | 7 mg-14 mg | 0.16 |
| sweetener: sucralose | 20 mg-40 mg | 0.44 |
| flavour enhancement agent | 180 mg-360 mg | 4.00 |
| colorant: beta-carotene | 20 mg-40 mg | 0.44 |
| sweetener: sorbitol | 810 mg-1620 mg | 18.02 |
| other additives | q.s. | q.s. |
| Total Weight | 45000 mg-9000 mg | 100 |

TABLE 2a

| Components | mg/dose (1-2 sachets) | % |
|---|---|---|
| hydrolysed collagen peptides | 2500 mg-5000 mg | 55.55 |
| Trans-resveratrol from fermentation of | 75 mg-150 mg | 1.67 |
| Saccharomyces cerevisiae (titrated to 90% | | |
| in trans-resveratrol) | | |
| Haematococcus pluvialis titrated to 2.5% in | 120 or 160 mg-240 or 320 mg | 3.55 |
| astaxanthin | (astaxanthin 3 or 4 mg 6 or 8 mg) | |
| vitamin C (ascorbic acid) | 500 mg-1000 mg | 13.33 |
| acidifier: citric acid | 100 mg-200 mg | 2.22 |
| anti-caking agent: silicon dioxide | 7 mg-14 mg | 0.16 |
| sweetener: sucralose | 20 mg-40 mg | 0.44 |
| flavour enhancement agent | 180 mg-360 mg | 4.00 |
| colorant: beta-carotene | 20 mg-40 mg | 0.44 |
| sweetener: sorbitol | 838 mg-1676 mg | 18.62 |
| other additives | q.s. | q.s. |
| Total Weight | 4500 mg-9000 mg | 100 |

(II) Compositions in Liquid Form of Suspensions (Phials).

The Compositions 3 and 4 are compositions according to the invention for oral use formulated in liquid form of water suspensions (packaged in phials) as reported in Table 3 and Table 4.

TABLE 3

| Components | mg/dose (1-2 phials) |
|---|---|
| hydrolysed collagen peptides | 2500 mg-5000 mg |
| resveratrol (98%) | 100 mg-200 mg |
| astaxanthin | 3 or 4 mg 6 or 8 mg |
| vitamin C (ascorbic acid)* | 500 mg-1000 mg |
| additives and/or excipients | q.s. |
| purified water | q.s. |
| Total Weight | 3830 mg/10 ml-7660 mg/20 ml |

*VNR %/1 phial = 1250

TABLE 4

| Components | mg/dose (1-2 phials) |
|---|---|
| hydrolysed collagen peptides | 2500 mg-5000 mg |
| resveratrol (90%) | 75 mg-150 mg |
| astaxanthin | 3 or 4 mg 6 or 8 mg |
| vitamin C (ascorbic acid)* | 500 mg-1000 mg |
| additives and/or excipients | q.s. |
| purified water | q.s. |
| Total Weight | 3800 mg/10 ml-7600 mg/20 ml |

*VNR %/1 phial = 1250

Specific examples of Compounds 3 and 4 are represented by Compounds 3a (Table 3a) and 4a (Table 4a), respectively, wherein astaxanthin, resveratrol and hydrolysed collagen peptides have the same technical characteristics as the products of Table 1a and 2a.

TABLE 3a

| Components | mg/dose (1-2 phials) | % |
|---|---|---|
| hydrolysed collagen peptides | 2500 mg-5000 mg | 25.00 |
| resveratrol from Polygunum cuspidatum | 100 mg-200 mg | 1.00 |
| (titrated to 98% in resveratrol) | | |
| Haematococcus pluvialis titrated to 2.5% in | 120 or 160 mg-240 or 320 mg | 1.60 |
| astaxanthin | (astaxanthin 3 or 4 mg 6 or 8 mg) | (0.09-0.10) |
| vitamin C: ascorbic acid | 500 mg-1000 mg | 5.00 |
| acidifier: citric acid | 100 mg-200 mg | 1.00 |
| preservative: sodium benzoate | 10 mg-20 mg | 0.10 |
| preservative: potassium sorbate | 5 mg-10 mg | 0.05 |
| sweetener: sucralose | 3 mg-6 mg | 0.03 |
| flavour enhancement agent | 180 mg-360 mg | 1.80 |
| colorant: beta-carotene | 20 mg-40 mg | 0.20 |
| purified water | q.s. | 64.22 |
| Total Weight | 3830 mg/10 ml-7660 mg/20 ml | 100 |

TABLE 4a

| Components | mg/dose (1-2 phials) | % |
|---|---|---|
| hydrolysed collagen peptides | 2500 mg-5000 mg | 25.00 |
| Trans-resveratrol from fermentation of | 75 mg-150 mg | 0.75 |
| Saccharomyces cerevisiae (titrated to 90% | | |
| in trans-resveratrol) | | |
| Haematococcus pluvialis titrated to 2.5% in | 120 or 160 mg-240 or 320 mg | 1.60 |
| astaxanthin | (astaxanthin 3 or 4 mg 6 or 8 mg) | (0.09-0.10) |
| vitamin C: ascorbic acid | 500 mg-1000 mg | 5.00 |
| acidifier: citric acid | 100 mg-200 mg | 1.00 |
| preservative: sodium benzoate | 10 mg-20 mg | 0.10 |
| preservative: potassium sorbate | 5 mg-10 mg | 0.05 |
| sweetener: sucralose | 3 mg-6 mg | 0.03 |
| flavour enhancement agent | 180 mg-360 mg | 1.80 |
| colorant: beta-carotene | 20 mg-40 mg | 0.20 |
| purified water | q.s. | 64.47 |
| Total Weight | 3800 mg/10 ml-7600 mg/20 ml | 100 |

### EXPERIMENTAL PART (II)

The aim of the project is to study the antioxidant and anti-inflammatory effects of a compound according to the invention comprising collagen, astaxanthin, trans-resveratrol and vitamin C (in short, mixture of the 4 substances or MIX), on an in vitro tendinopathy model.

The model is based on the use of primary human tenocytes, obtained through isolation from Achilles tendon, purchased from Zen-Bio (TEN-F), cultured and used according to the supplier's instructions. The inflammation model was obtained by treating the cells with IL1β (10 ng/ml, 0/N).

The oxidative stress model was obtained by treating the cells with H₂O₂ (1.5 mM, 2 hours)

### 1. Solubility Studies

The individual substances, received in powder form, were dissolved as follows:
Vitamin C: vitamin C was dissolved in water, by preparing a 100 mM stock (corresponding to 17.612 mg/ml).
Collagen peptides: collagen peptides have a low molecular weight and a hydrophilic nature which allowed to prepare a stock equal to 100 mg/ml.
Astaxanthin (AstaReal EL25): astaxanthin was dissolved in DMSO and a stock equal to 100 mg/ml was prepared. The stock concentration was established so that the final dose of the treatment contains a percentage of DMSO less than 1%, otherwise toxic to cells.

Note: the formulation contains 2.5% astaxanthin. From here on, the concentrations indicated for the treatments do not refer to the concentration of the raw material AstaREAL EL25, but to the actual concentration of astaxanthin (2.5%).
Resveratrol (CWD90 EVOLVA): resveratrol was dissolved in DMSO and a stock equal to 50 mg/ml was prepared. The stock concentration was established so that the final dose of the treatment contains a percentage of DMSO less than 1%, otherwise toxic to cells.

### 2. Cytotoxicity Studies—MTT Test

Preliminary cytotoxicity studies (i.e. MTT test (3-(4,5-dimethylthiazol-2-yl)-2,5-diphenyltetrazolium bromide) and the subsequent statistical analysis) were conducted on healthy tenocytes treated with the individual substances in order to identify the doses to be used for subsequent experiments, as well as the doses to be mixed to analyse synergism.

Cells were seeded in 48 well multiwells, at a density of 6000 cells/well. After 48 ore, they were treated with different concentrations of vitamin C (from 0.88 μg/mL to 352 μg/mL), collagen peptides (from 100 μg/mL to 3000 μg/mL), astaxanthin (from 1 μg/mL to 1000 μg/mL) or resveratrol (from 0.1 μg/mL to 100 μg/mL) for 24 hours. The range of concentrations selected for each substance was determined based on the data reported in literature. The MTT test was conducted at the end of the treatment. Briefly, the growth medium was removed and replaced with phenol red-free medium, containing 0.5 mg/ml MTT (3-(4,5-dimethylthiazol-2-yl)-2,5-diphenyltetrazolium bromide), and the cells were placed in an incubator for one hour. The conversion of yellow MTT to purple formazan by mitochondrial enzymes indicates mitochondrial activity and therefore cell viability. The formazan deposits in the cells were then solubilised with isopropanol, obtaining a homogeneous solution whose absorbance can thus be measured at the spectrophotometer (absorbance at 510 nm).

The statistical analysis was conducted using the GraphPad Prism program, using one-way ANOVA, followed by Dunnet tests for multiple comparisons.

The results obtained (not reported) show that, at the tested doses, collagen and astaxanthin are devoid of toxicity, while a negative effect on cell viability is observed by treating the tenocytes with vitamin C (352 μg/ml) or resveratrol (50 and 100 μg/ml).

Based on said cytotoxicity results, the subsequent experiments were conducted with the following doses (obtained by means of 3 serial dilutions 1:2):
Vitamin C: 88, 44, 22, 11 μg/ml (i.e. 1 and dilution 1:2, 1:4, and 1:8)
Collagen: 1000, 500, 250, 125 μg/ml
Astaxanthin: 500, 250, 125, 62.5 ng/ml
Resveratrol: 10, 5, 2.5, 1.25 μg/ml.

### 3. Cytotoxicity Tests—Combination (According to the Invention)

Similar cytotoxicity tests were thus conducted to check for possible toxicity due to the combination of the substances. The mixture of the 4 compounds (MIX) was prepared by combining the maximum doses of each substance (i.e. vitamin C 88 μg/ml, collagen 1000 μg/ml, astaxanthin 500 ng/ml, resveratrol 10 μg/ml) and the subsequent concentrations are obtained using three serial dilutions 1:2 (1:2, 1:4, 1:8, respectively).

The MTT test and subsequent statistical analysis were conducted as described in paragraph 2 and it was found (results not reported) that the combination of the four substances, at each of the tested concentrations, has no toxic effects on the tenocytes.

### 4. Cytotoxicity Tests in the Presence of H₂O₂

The ability of the substances under analysis to combat oxidative stress was evaluated.

The tenocytes were treated with H₂O₂, known inducer of radical oxygen species (ROS) and the ensuing oxidative stress, in the presence or in the absence of the individual substances or of the mixture thereof (MIX, compound according to the invention).

To confirm that the concentration of H₂O₂ (and the combination thereof with the substances) was not toxic, and that consequently the production of ROS was not related to cell death, an MTT test was conducted on the tenocytes treated according to the following experimental scheme.

The tenocytes were seeded in 48-well multiwells, at the density of 6000 cells each. After 48 hours, a pre-treatment was conducted with different concentrations of the individual substances, or of the mixture thereof. After the 24 hours of treatment, 1.5 mM H₂O₂ was added to the medium, for 2 hours, and the MTT test was subsequently conducted as described in paragraph 2. The statistical analysis was conducted using the GraphPad Prism program, using one-way ANOVA, followed by Tukey tests for multiple comparisons.

The results obtained (not reported) show that H₂O₂ per se does not affect the viability of the tenocytes. Similarly, the combination thereof with the four substances or the mixture thereof (MIX) does not cause a toxic effect on the tenocytes themselves.

### 5. Analysis of the Production of Radical Oxygen Species (ROS)

The efficacy of the individual substances and the combination thereof (MIX, compound according to the present invention) in combating the production of radical oxygen species (ROS) in the oxidative stress model was analysed.

The tenocytes were seeded in 96-well black multiwells, at a density of 3000 cells each. After 48 hours, a pre-treatment was conducted with different concentrations of the individual substances, or of the mixture thereof (MIX)(Table 5). After 24 hours from the treatment, the H₂DCFDA (2′,7′-dichlorodihydrofluorescein diacetate) probe was added to the growth medium, which is cleaved by enzymes involved in oxidative stress. The product of this conversion, DCF (dichlorofluorescein), is capable of absorbing light at a λₑₓ 492-495 nm, and reemitting at a λₑₘ 517-527 nm. After one hour of incubation, 1.5 mM H₂O₂ was further added to the medium for 2 hours, and ROS production was measured by means of spectrophotometry.

### 5.1. EXPERIMENTAL DESIGN SCHEME

Independent variables:
Treatment 1. Compounds administered: factor in 6 levels: Astaxanthin, Collagen, Resveratrol, Vitamin C,

Mix and an Untreated Control
Treatment 2. Addition of hydrogen peroxide (H₂O₂): factor in two levels: YES/NO
Concentration. Concentration of treatments 1. Factor in 4 levels: C1, C2, C3, C4 (Table 5)
Response variable: fluorescence.

TABLE 5

| Treatment | C1 | C2 | C3 | C4 |
|---|---|---|---|---|
| Astaxanthin | 500 ng/ml | 250 ng/ml | 125 ng/ml | 62.5 ng/ml |
| Collagen | 1000 ug/ml | 500 ug/ml | 250 ug/ml | 125 ug/ml |
| Resveratrol | 10 ug/ml | 5 ug/ml | 2.5 ug/ml | 1.25 ug/ml |
| Vitamin C | 88 ug/ml | 44 ug/ml | 22 ug/ml | 11 ug/ml |
| Mix | 1:1 | 1:2 | 1:4 | 1:8 |

### 5.2. Objective of the Analysis

The objectives of the statistical analysis of the data of the trial in question were:
1. Comparison of the response (fluorescence) between treatment 1 and control in the presence and in the absence of hydrogen peroxide in each of the 4 concentrations (C1-C4).
2. Comparison of the response (fluorescence) between Mix and the other treatments 1 in the presence and in the absence of hydrogen peroxide in each of the 4 concentrations (C1-C4).
3. Comparison of individual treatments and Mix at 4 concentrations (C1-C4) in the group of samples treated with hydrogen peroxide only.

### 5.3. Statistical Analysis

Analysis 1 (objectives 1 and 2): the most suitable statistical analysis for this type of trial is a classification multi-way analysis of variance model (ANOVA) in which the fluorescence value (after correction for the respective blanks) represents the dependent variable. Treatments 1 and 2 together with their interaction represent the independent variables. An independent analysis was conducted for each concentration level.

Analysis 2 (objective 3): in this case, a one-way analysis of variance model in which the fluorescence value (after correction for the respective blanks) represented the dependent variable and the treatment (individual treatments at a given concentration and mix treatment at a lower concentration) represented the independent variable was used.

### 5.4. Results

For the sake of simplicity, only the data for the tested concentration C4 are reported in detail below. Similar results were obtained for the tested concentrations C1-C3.

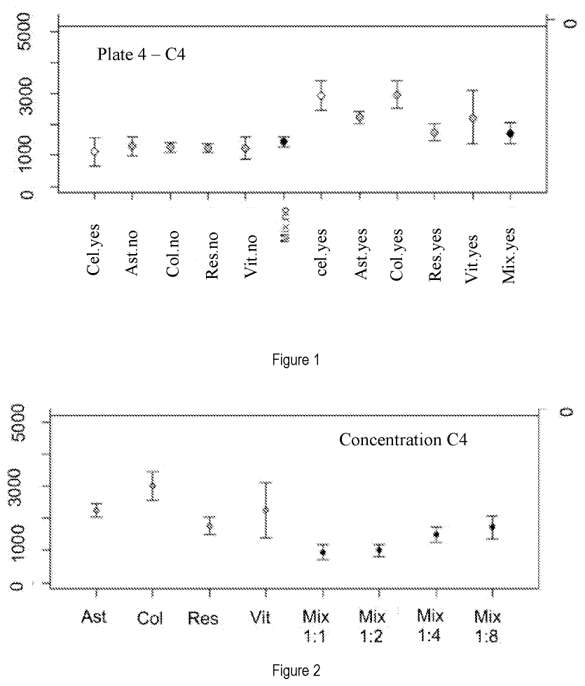

FIG. 1 shows the comparison of the fluorescences between treatments in the presence and in the absence of hydrogen peroxide at concentration C4.

FIG. 2 shows the difference in the response of the individual treatments at concentration C4 with respect to the four treatments in mixture (1:1, 1:2, 1:4 and 1:8 MIX).

As observable from FIG. 1, the results obtained suggest that treatment with H₂O₂ is capable of efficiently inducing ROS production, corroborating the experimental model: fluorescence increases significantly following treatment with hydrogen peroxide and the difference between the two controls (with and without hydrogen peroxide) is highly significant.

FIG. 1 shows that the mixture of the four substances (MIX, compound according to the invention) is capable of combating the ROS production with respect to the oxidised control (i.e. control in the presence of H₂O₂ (acronym, cel.si)), demonstrating efficacy already at the lowest concentration (1:8 MIX) and a greater effectiveness with respect to the individual components of the mixture with similar concentration, in particular with respect to collagen.

In the light of the results of the statistical analysis conducted (FIG. 2), it can be concluded that, since there is no statistically significant difference between the action of the 1:1 and 1:2 MIX, a halved dosage is sufficient to have the same biological efficacy as that demonstrated by the MIX with higher concentration (i.e. 1:1 MIX). Likewise, the use of an eight-fold decreased concentration (1:8 MIX) shows the same efficacy as the four-fold decreased concentration (MIX 1:4). Furthermore, the effect of the 1:8 MIX is greater than that of the individual components (in a statistically significant manner with respect to astaxanthin and collagen).

Based on what is reported above, it can be concluded that
the effect of the 1:8 MIX (where the concentration of the active components is decreased by one eighth) can be compared to that obtained with the 1:4 MIX and not much lower with respect to the one of the 1:1 MIX (such as a mixture of the substances at the reference concentration C1), allowing to use low dosages of the individual components in mixture (1:8 Mix versus 1:1 Mix) to obtain similar effects (synergistic effect); and
the effect of the 1:8 MIX is greater with respect to the effect of the individual components of the mixture (in particular collagen and astaxanthin) at a similar concentration (C4).

### 6. Cytotoxicity Tests in the Presence of IL-1β

In order to evaluate the ability of the substances to combat the inflammatory process, the tenocytes were treated with IL-1β, a known inflammatory inducer, in the presence or in the absence of the individual substances or of the mixture thereof. To confirm that the concentration of IL-1β (and the combination thereof with the substances) was not toxic, an MTT test was conducted on the tenocytes treated according to the following experimental scheme.

The tenocytes were seeded in 48-well multiwells, at the density of 6000 cells each, and after 32 hours were treated with IL-1β 10 ng/ml O/N, with the aim of inducing the inflammatory process. The subsequent day, the cells were treated with different dosages of the individual substances, or of the combination thereof, for 24 hours. Lastly, the MTT test was conducted as described above (paragraph 2).

The statistical analysis was conducted using the GraphPad Prism program, using one-way ANOVA, followed by Tukey tests for multiple comparisons.

The results (not reported) show that IL-1β has no toxic effects on the tenocytes, at the dose used. At the same time, it can be observed that no toxic effect is induced by the combination thereof with the four substances, and the mixture thereof.

### 7. Dosage of Markers of Inflammation and Tissue Remodelling

Once the absence of toxicity in the presence of IL-1β was established, the efficacy of the individual substances, and the combination thereof, in decreasing the secretion of inflammation mediators and in altering the production of tissue remodelling mediators (IL-6 and MMP-2) was analysed.

To this end, the tenocytes were seeded in 6-well multiwells, at a density of 60000 cells each. After 32 hours, the inflammatory process was induced by treating with IL-1β 10 ng/ml, 0/N. The subsequent day, the cells were treated with different concentrations of the individual substances, or of the combination thereof (MIX). After 24 hours of treatment, the conditioned medium of the tenocytes was harvested, aliquoted, and stored for subsequent analysis. The secretion of inflammation mediators or of the tissue remodelling was evaluated using ELISA tests on the conditioned medium, following the protocols indicated on the respective datasheets. The analysed molecules are: IL-6 and MMP-2.

### 7. A. Modulation Analysis of IL-6

### 7.A1. EXPERIMENTAL DESIGN SCHEME

Experimental design similar to what is reported in paragraph 5.1 wherein IL-1β is added to “Treatment 2” instead of H₂O₂.

Response variable: absorbance.

### 7.A2. Objective of the Analysis

The objectives of the statistical analysis of the data of the trial in question were:

1. Comparison of the response (expression of IL-6 measured as absorbance) between treatment 1 and control in the presence and in the absence of IL-1β.

2. Comparison between individual and mix treatments at the 4 concentrations in the presence of IL-1β.

### 7.A3. Statistical Analysis

The most suitable statistical analysis for this type of trial is a classification multi-way analysis of variance model (ANOVA) in which the absorbance value (after correction for the respective blanks) represents the dependent variable. Treatments 1 and 2 together with their interaction represent the independent variables. An independent analysis was conducted for each concentration level.

### 7.A4. Results

For the sake of simplicity, only the data for the tested concentration C4 are reported in detail below. Similar results were obtained for the tested concentrations C1-C3.

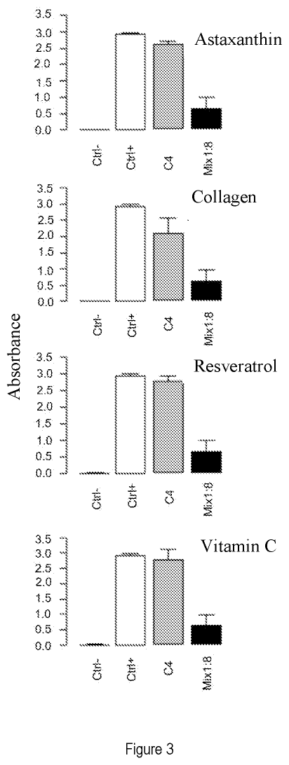

FIG. 3 shows the absorbances (expression index IL-6) of the controls in the presence and in the absence of IL-1β, of the individual components at concentration C4 and of the mixture thereof (1:8 MIX) in the presence of IL-1β.

As observable from FIG. 3, the inflammatory process was efficiently induced by treatment with IL-1β (comparison of untreated and treated positive control).

Furthermore, the mixture of the four substances at concentration C4 (1:8 MIX, compound according to the invention) is capable of decreasing IL-6 expression in a statistically significant manner, when compared with positive control (treated with IL-1β) and with the individual active components at concentration C4 (FIG. 3).

Thus, it can be concluded that the effect on the decrease in the level of inflammation of the mixture according to the invention is synergistic with respect to those of the active components taken individually.

### 7. B. Modulation Analysis of MMP-2

### 7.B1. EXPERIMENTAL DESIGN SCHEME

Experimental design similar to what is reported in paragraph 5.1 wherein IL-1β is added to “Treatment 2” instead of H₂O₂.

Response variable: absorbance.

### 7.B2. Objective of the Analysis

The objectives of the statistical analysis of the data of the trial in question were:
1. Comparison of the response (expression of MMP-2 measured as absorbance) between treatment 1 and control in the presence and in the absence of IL-1β.
2. Comparison between individual and mix treatments at the 4 concentrations in the presence of IL-1β.

### 7.B3. Statistical Analysis

The most suitable statistical analysis for this type of trial is a classification multi-way analysis of variance model (ANOVA) in which the absorbance value (after correction for the respective blanks) represents the dependent variable. Treatments 1 and 2 together with their interaction represent the independent variables. An independent analysis was conducted for each concentration level.

### 7.B4. Results

For the sake of simplicity, only the data for the tested concentration C4 are reported in detail below. Similar results were obtained for the tested concentrations C1-C3.

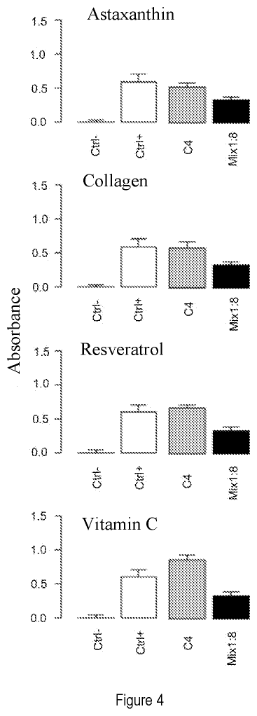

FIG. 4 shows the absorbances (expression index MMP-2) of the control in the presence and in the absence of IL-1β, of the individual components at concentration C4 and of the mixture thereof (1:8 MIX) in the presence of IL-1β.

As observable from FIG. 4, the pro-inflammatory stimulus induced by IL-1β induces an increase in the secretion of the MMP-2 enzyme (comparison of the untreated or treated positive control with IL-1β). Furthermore, the data reported in FIG. 4 shows that the mixture of the four substances at concentration C4 (1:8 Mix, compound according to the invention) is capable of decreasing the production of MMP-2 in a statistically significantly manner when compared with positive control (treated with IL-1β) and all active components administered individually at concentration C4.

A synergistic effect of the mixture of the four substances (compound according to the invention) with respect to the individual active substances in decreasing the secretion of the MMP-2 enzyme is therefore evident.

1. A composition comprising:
(I) a mixture M comprising, or alternatively, consisting of:
(a) collagen or collagen peptides;
(b-i) resveratrol;
(c-i) astaxanthin;
(d) ascorbic acid;
and, optionally, said composition comprises
(II) at least one pharmaceutical or food grade additive and/or excipient;
wherein said composition is for use in a method for the preventive and/or curative treatment of tendinopathy, enthesopathy, peritendinitis, tendinosis, tendon rupture, tenosynovitis, and joint inflammation and/or joint pain, in a subject in need.

2. The composition for use according to claim 1, wherein said (a) collagen peptides are hydrolysed collagen peptides; preferably (a) enzymatically hydrolysed collagen peptides.

3. The composition for use according to claim 1, wherein said (a) collagen or collagen peptides have an average molecular weight comprised in the range from 0.3 kDa to 10 kDa, preferably from 1 kDa to 5 kDa, more preferably from 1.5 kDa to 3 kDa.

4. The composition for use according to claim 1, wherein said (b-i) resveratrol consists of (b-i.i) an extract of Polygonum cuspidatum titrated in resveratrol in a percentage by weight comprised from 80% to 99.5% with respect to the total weight of the extract, preferably from 90% to 99.5%, more preferably about 98%; preferably wherein said (b-i.i) is a dry root extract of Polygonum cuspidatum.

5. The composition for use according to claim 1, wherein said (b-i) resveratrol consists of (b-i.ii) a resveratrol from fermentation of Saccharomyces cerevisiae comprising resveratrol in a percentage comprised from 70% to 99.5% with respect to the total weight of resveratrol from fermentation, preferably from 85% to 95%, more preferably at about 90%.

6. The composition for use according to claim 1, wherein said (c-i) astaxanthin consists of:
(c-i.i) extract of Haematococcus pluvialis titrated in astaxanthin in a percentage by weight comprised from 0.1% to 10% with respect to the total weight of extract, preferably from 0.5% to 7.5%, more preferably from 1% to 5%.

7. The composition for use according to claim 1, wherein said mixture M comprises at by weight percentages with respect to the total weight of the mixture M consisting of (a), (b), (c) and (d):
(a) collagen or collagen peptides in a percentage comprised from 50% to 90%, preferably from 75% to 85%;
(b) resveratrol in a percentage comprised from 0.5% to 8%, preferably from 2% to 4%;
(c) astaxanthin in a percentage comprised from 0.05% to 2%, preferably from 0.05% to 0.5%;
and (d) L-ascorbic acid in a percentage comprised from 5% to 30%, preferably from 12% to 18%.

8. The composition for use according to claim 1, wherein a dosage unit of said composition comprises at the following by weight ratios [(a) collagen or collagen peptides from 500 to 50]: [(b) resveratrol from 5 to 0.5]: [(c) astaxanthin from 0.25 to 0.025]: [(d) L-ascorbic acid from 50 to 5];
preferably [(a) collagen or collagen peptides from 200 to 50]: [(b) resveratrol from 2 to 0.5]: [(c) astaxanthin from 0.1 to 0.025]: [(d) L-ascorbic acid from 20 to 4];
more preferably [(a) collagen or collagen peptides 100]: [(b) resveratrol 1]: [(c) astaxanthin from 0.05]:
[(d) L-ascorbic acid 8.5-9].

9. The composition for use according to claim 1, wherein said composition is formulated for oral use.

10. The composition for use according to claim 9, wherein said composition is in powdered or granular solid form or, alternatively, in liquid form of suspensions or solutions.

11. A method of treating tendinopathy, enthesopathy, peritendinitis, tendinosis, tendon rupture, tenosynovitis, joint inflammation and/or joint pain comprising administering to a patient in need thereof a composition comprising:
(I) a mixture M comprising, or alternatively, consisting of:
(a) collagen or collagen peptides;
(b-i) resveratrol;
(c-i) astaxanthin;
(d) ascorbic acid;
and, optionally, said composition comprises
(II) at least one pharmaceutical or food grade additive and/or excipient.

12. The method of claim 11, wherein said (a) collagen peptides are hydrolysed collagen peptides;
preferably (a) enzymatically hydrolysed collagen peptides.

13. The method of claim 11, wherein said (a) collagen or collagen peptides have an average molecular weight comprised in the range from 0.3 kDa to 10 kDa, preferably from 1 kDa to 5 kDa, more preferably from 1.5 kDa to 3 kDa.

14. The method of claim 11, wherein said (b-i) resveratrol consists of (b-i.i) an extract of Polygonum cuspidatum titrated in resveratrol in a percentage by weight comprised from 80% to 99.5% with respect to the total weight of the extract, preferably from 90% to 99.5%, more preferably about 98%;
preferably wherein said (b-i.i) is a dry root extract of Polygonum cuspidatum.

15. The method of claim 11, wherein said (b-i) resveratrol consists of (b-i.ii) a resveratrol from fermentation of Saccharomyces cerevisiae comprising resveratrol in a percentage comprised from 70% to 99.5% with respect to the total weight of resveratrol from fermentation, preferably from 85% to 95%, more preferably at about 90%.

16. The method of claim 11, wherein said (c-i) astaxanthin consists of:
(c-i.i) extract of Haematococcus pluvialis titrated in astaxanthin in a percentage by weight comprised from 0.1% to 10% with respect to the total weight of extract, preferably from 0.5% to 7.5%, more preferably from 1% to 5%.

17. The method of claim 11, wherein said mixture M comprises at by weight percentages with respect to the total weight of the mixture M consisting of (a), (b), (c) and (d):
(a) collagen or collagen peptides in a percentage comprised from 50% to 90%, preferably from 75% to 85%;
(b) resveratrol in a percentage comprised from 0.5% to 8%, preferably from 2% to 4%;
(c) astaxanthin in a percentage comprised from 0.05% to 2%, preferably from 0.05% to 0.5%;
and (d) L-ascorbic acid in a percentage comprised from 5% to 30%, preferably from 12% to 18%.

18. The method of claim 11, wherein a dosage unit of said composition comprises at the following by weight ratios [(a) collagen or collagen peptides from 500 to 50]: [(b) resveratrol from 5 to 0.5]: [(c) astaxanthin from 0.25 to 0.025]: [(d) L-ascorbic acid from 50 to 5];
preferably [(a) collagen or collagen peptides from 200 to 50]: [(b) resveratrol from 2 to 0.5]: [(c) astaxanthin from 0.1 to 0.025]: [(d) L-ascorbic acid from 20 to 4];
more preferably [(a) collagen or collagen peptides 100]: [(b) resveratrol 1]: [(c) astaxanthin from 0.05]:
[(d) L-ascorbic acid 8.5-9].

19. The method of claim 11, wherein said composition is formulated for oral use.

20. The method of claim 19, wherein said composition is in powdered or granular solid form or, alternatively, in liquid form of suspensions or solutions.
